(19) United States
(12) Patent Application Publication
(10) Pub. No.: US 2006/0161215 A1
(43) Pub. Date: Jul. 20, 2006

(54) Weld plate contact for implanted medical devices

(76) Inventor: Jacques Naviaux, Rancho Palos Verdes, CA (US)

Correspondence Address: WALTER A. HACKLER, Ph.D.;PATENT LAW OFFICE, SUITE B, 2372 S.E. BRISTOL STREET, NEWPORT BEACH, CA, 92660-0755, US

(21) Appl. No.: 11/332,136

(22) Filed: Jan. 13, 2006

Related U.S. Application Data
(60) Provisional application No. 60/644,952, filed on Jan. 18, 2005.

(51) Int. Cl. A61N 1/375

(52) U.S. Cl. 607/37

(57) ABSTRACT

A weld plate contact assembly is provided for implanted medical devices which includes at least one molded plastic housing having two halves joined together with the halves defining a bore therethrough. A canted coil spring is disposed within the housing halves along a bore circumference and includes a pigtail lead which extends to an exterior of the housing halves. A weld plate overlays the housing halves exterior which is joined to both the pigtail lead and devices wires.

The present application claims priority from U.S. Provisional Application Ser. No. 60/644,952 filed Jan. 18, 2005 which is incorporated in its entirety into the present application.

This invention generally relates to implantable medical devices and is more particularly directed to the electrical conductive path between a pulse generator and an implantable lead or leads.

Implantable medical electronics devices typically consist of an implanted pulse generator for providing electrical stimulation to targeted tissues and an implantable lead or leads that are used to transmit electrical impulse to the targeted tissues. Early devices were developed for cardiac pacemaking, and now such devices have a number of applications for cardiac rhythm management, treatment for congestive heart failure, and implanted defibrillators. Other devices are used for neuromodulation with a wide range of uses such as pain control, nervous tremor mitigation, incontinence treatment, epilepsy seizure reduction, vagus nerve stimulation for clinical depression and others. This rapidly growing field will undoubtedly have even wider application in the future.

In general, such devices include an implanted impulse generator that may also be capable of sensing body activity such as an irregular heart-beat. The impulse generator may generate an electrical impulse or signal that is transmitted to a targeted tissue or tissues or nerve area or areas through an implanted lead. Once the lead or leads are implanted in the body, removal may involve major surgery with an attendant risk factor. Therefore, a reliable method of connecting and disconnecting the leads is required since the implanted impulse generator may have to be replaced to update the unit or to replace the battery.

These devices are hermetically sealed and include circuitry and a power supply. Current practice is to place a molded header containing a connector on top of the unit to provide a means of housing the electrical contacts for the leads. While some applications are very simple requiring only two leads because they only have to transmit two discrete signals to the tissues, others are very complex and require a very large number of discrete electrical impulses. Each electrical impulse requires a discrete conductive path between the impulse generator and the implanted lead.

Several different types of contacts are now in use ranging from setscrews to various types of spring contacts. These contacts are embedded in the connector which is generally made of a silicon filled implantable polymeric. The lead generally consists of a series of conductive rings separated by insulative spaces so that when it is fully inserted into the header, each conductive ring is placed in contact with a connector contact. Each contact in turn has to be connected to a discrete lead from the impulse generator.

In current practice, the connector generally consists of a setscrew in a metal connector or some type of spring in a metal housing. Where the spring is used, it provides the conductive path between the metal housing and the conductive ring of the lead. Setscrews are very undesirable where large numbers of connectors are required because each contact must be tightened with a torque wrench. A spring retained in a metal housing provides a reliable contact with controlled insertion forces that is convenient for both insertion and removal and obviates the requirement for a torque wrench. A canted coil spring has very desirable characteristics for this application since its nearly constant force over a wide range of deflection compensates for any irregularities on the surfaces of the lead electrical contact rings and the insertion force can be controlled.

The housings, which can number anywhere from two to twenty-four or even more are now machined from metals such as MP35N, titanium, or even platinum, are significant cost drivers. Therefore, it is highly desirable to utilize an implantable polymeric biocompatible material for the housing that can be fabricated by injection molding to reduce the cost of the contacts. However, an electrical path must be added to the plastic housing.

The present invention utilizes a spring with a pigtail for providing the electric path which is connected to the lead from the pulse generator. The housing is molded from an implantable polymeric material in two pieces with the pigtail spring leads extending from the housing. Various techniques may be used to attach the pigtail leads to the leads from the impulse generator. This connection method minimizes contact resistance and provides for a very robust electrical contact by using a weld plate as an electrical bus.

### SUMMARY OF THE INVENTION

A weld plate contact assembly in accordance with the present invention suitable for implantable medical devices and generally includes at least one molded plastic housing including two halves joinable together with the joined halves defining a bore therethrough. The use of molded plastic for the housing enables fabrication by injection molding which significantly reduces the cost of the assembly.

A canted coil spring is disposed within the housing halves along a bore circumference with the spring including a pigtail lead that extends to an exterior of the housing halves.

A weld plate is provided which overlays the housing halves exterior and is joined at the pigtail lead. In addition, at least one device wire is also joined to the weld plate.

More particularly, the plastic housing halves include means for enabling aligned engagement with adjacent housing halves with the alignment including coaxially aligned bores.

More specifically, the means for aligned engagement may include opposing pins extending from each housing half which, upon engagement, with adjacent housing halves provides for the coaxially aligned bores.

The assembly may include a plurality of molding plastic housings engaged with one another and aligned with coaxial bores with each of the plurality including a canted coil spring with a pigtail lead extending to an exterior of a corresponding housing and joined to a corresponding weld plate.

### BRIEF DESCRIPTION OF THE DRAWINGS

The advantages and features of the present invention will be better understood by the following description when considered in conjunction with the accompanying drawings, in which:

FIG. 1 is a plan view of a generator illustrating a header which includes a plurality of molded plastic housings each having joined halves for defining a bore therethrough along with an implantable lead;

FIG. 2 is a plan view of the implantable lead shown in FIG. 1 illustrating spaced apart conductive rings;

FIG. 3 is a an exploded perspective view of housing pairs joined both through pins for aligning a bore therethrough along with a canted coil spring disposed within the housing bores and having a pigtail lead extending to a weld plate; and

FIG. 4 is a side view of a housing halve with the canted coil spring disposed therein more clearly illustrating attachment of the spring pigtail to the weld plate along with a device wire joined to the weld plate.

### DETAILED DESCRIPTION

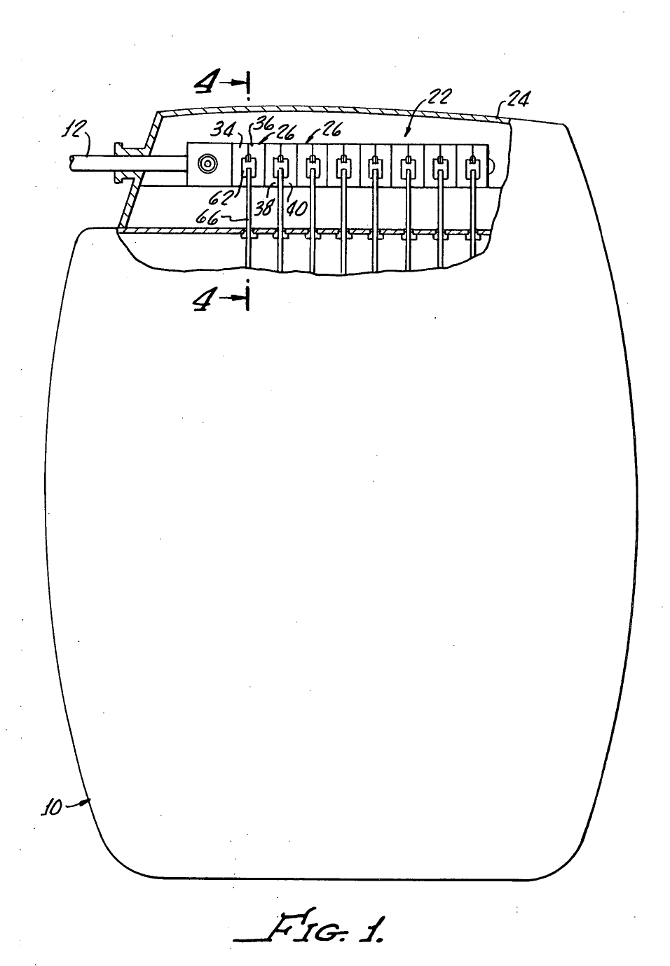

With reference to FIG. 1, there is shown an impulse generator 10 for generating electrical pulses that deliver to target tissue (not shown) by an implantable lead 12.

As illustrated in FIG. 2, the implanted lead 12 includes a plurality of spaced apart conductive rings 14 separated by insulators 18.

It should be appreciated that the generator 10 may also be a receptor and processor of information from the target tissues through the implanted lead 12.

As hereinabove briefly noted, the generator 10 includes a wide range of uses such as cardiac rhythm management, implanted defibrillators, and neurostimulators used for the control of pain, treatment of nervous disorders, incontinence, clinical depression, and other applications. However, the fundamental requirements are generally the same for all of these applications. That is, an electrical signal must be transmitted from the generator 10 to the lead 12 and thereafter to the target tissue. Conversely, the generator may, in fact, act as a receiver for gathering information from the targeted tissue through the implanted lead 12.

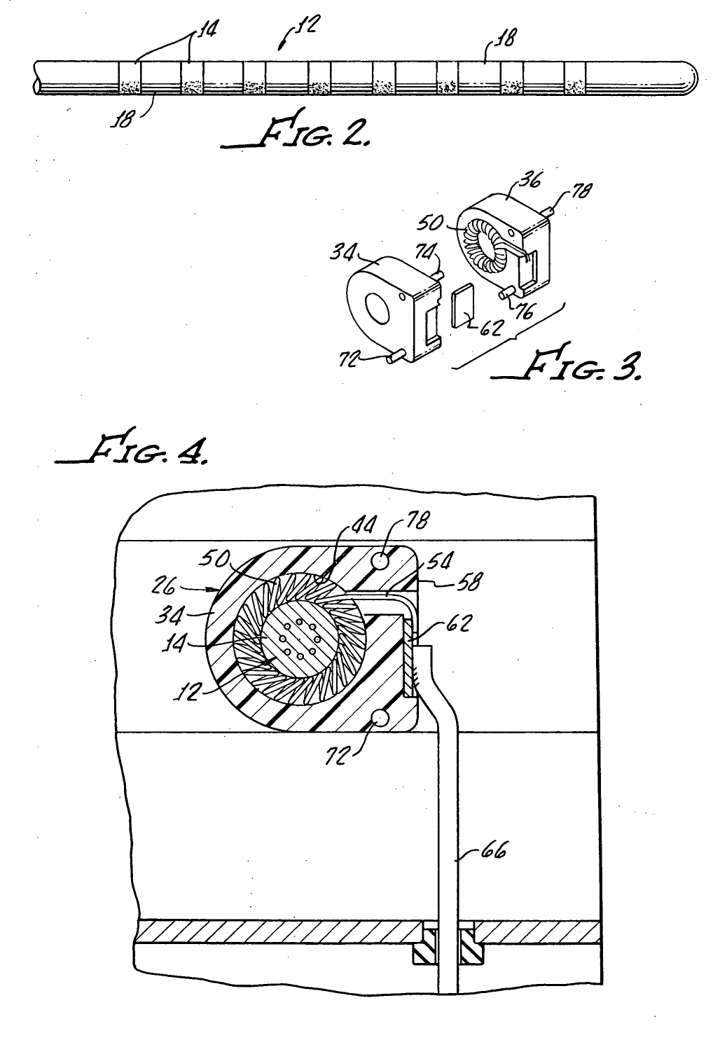

As illustrated in FIGS. 3 and 4 an electrical path is provided by a weld plate contact assembly 22 disposed in a header 24 (FIG. 1) which includes molded housings 26, 28 formed from a polymeric biocompatible material suitable for fabrication by injection molding.

The housings 26, 28 include housing halves 34, 36, 38, 40 respectively which are adjoined together to form a bore 44 therethrough, see FIG. 4. A canted coil spring 50 is disposed within the housing halves 34, 36 and includes a pigtail lead 54 extending to an exterior 58 of the housing 26 which is joined to a weld plate 62 which overlays the housing halves exterior 58.

At least one device wire 66 is also joined to the weld plate for providing electrical continuity between the impulse generator 10 and a conductive ring through the spring 50, pigtail 54, and weld plate 62.

As illustrated, the housings 26 with the housing halves 34, 36, 38, 40 are aligned with one another to define the bore 44 into which the implantable lead 12 is inserted.

This alignment is enabled through the use of pins 72, 74, 76, 78 opposing one another and extending from housing halves 34, 36 which provide a means for enabling alignment of adjacent housing halves 34, 36 with the coaxial bore 44. It should be appreciated that other structure may be provided in addition to or in lieu of the pins 72, 74, 76, 78 for enabling the aligned engagement of the housing halves 34, 36.

It should be appreciated that the present invention is directed to a contact assembly which is modular in concept and accordingly any number of electrical contacts may be provided. The lead 66 from the impulse generator 10 transmits electrical impulse from the generator 10 to the implanted lead 12 through the contact assembly 22 as shown there is multiplicity of contacts with each contact carrying a discrete signal. The number of contacts can vary from two to twenty-four or higher and the electrical pulse travels from the impulse generator 10 through the contact assembly 22 through the pigtail lead 54 and spring 50 to the contact rings 14.

The weld plate 62 provides for a robust electrical connection despite the use of a polymeric housings 26, 28.

Although there has been hereinabove described a specific weld plate contact for implanted medical devices in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. A weld plate contact assembly for implanted medical devices, the contact assembly comprising:
at least one molded plastic housing including two halves joined together, the joined halves define a bore therethrough;
a canted coil spring disposed within the housing halves along a bore circumference and including a pigtail lead extending to an exterior of the housing halves;
a weld plate overlaying the housing halves exterior and joined to the pigtail lead; and
at least one device wire joined to said weld plate.

2. The assembly according to claim 1 wherein each of the plastic housing halves include means for enabling aligned engagement with adjacent plastic housing halves, the aligned engagement including coaxially aligned bores.

3. The assembly according to claim 2 wherein the means for aligned engagement comprises opposing pins extending from each housing half.

4. The assembly according to claim 3 further comprising a plurality of molded plastic housings engaged with one another and aligned with coaxially bores each of the plurality including a canted coil spring with a pigtail lead extending to an exterior of the corresponding housing and joined to a corresponding weld plate.

5. The assembly according to claim 4 further comprising a plurality of device wires, each device wire joined to a corresponding weld plate.

6. The assembly according to claim 4 further comprising an implantable lead insertable into the coaxial bores and having spaced apart conductive rings alignable with corresponding canted coil springs.

7. A weld plate contact assembly for implanted medical devices, the contact arm comprising:
a molded plastic housing including two halves joined together;
a canted coil spring disposed inside the housing and having spring pigtail bores extending to an exterior of the two halves of the housing;
a weld plate overlaying the housing two halves exterior and joined to the pigtail lead; and
a device wire joined to the weld plate.

8. The assembly according to claim 7 wherein each of the housing halves includes means for enabling engagement with adjacent housing halves, the aligned engagement including coaxially aligned bores.

9. The assembly according to claim 8 wherein the means for aligned engagement comprises opposing pins extending from each housing half.

10. The assembly according to claim 9 further comprising a plurality of device wires, each device wire joined to corresponding weld plates.

11. The assembly according to claim 10 further comprising an implantable lead insertable into the coaxial bores and having spaced apart conductive rings alignable with each of the canted coil springs.
